(19) United States
(12) Patent Application Publication
(10) Pub. No.: US 2025/0272650 A1
(43) Pub. Date: Aug. 28, 2025

(54) EXPERIMENTAL ANIMAL MANAGING METHOD

(71) Applicant: CHUNGNAM NATIONAL UNIVERSITY HOSPITAL, Daejeon (KR)

(72) Inventor: Kwang Yeon CHOI, Daejeon (KR)

(73) Assignee: CHUNGNAM NATIONAL UNIVERSITY HOSPITAL, Daejeon (KR)

(21) Appl. No.: 18/250,412

(22) PCT Filed: Oct. 25, 2021
(86) PCT No.: PCT/KR2021/014979

§ 371 Date: Aug. 29, 2023

(30) Foreign Application Priority Data
Oct. 26, 2020 (KR) 10-2020-0139309

(51) Int. Cl. A01K 29/00; A01K 1/03; G06Q 10/087

(52) U.S. Cl. CPC G06Q 10/087

(57) ABSTRACT

The present invention relates to an experimental animal managing method. Disclosed is an experimental animal managing method comprising: a receiving step (S10) of receiving user log data and individual data of an experimental animal; and a processing step (S20) of calculating at least one of an expected production amount of individuals, the expected number of cages to be produced, and the expected total number of cages, on the basis of the user log data and the individual data, and optimizing at least one among the calculated expected production amount of individuals, the calculated expected number of cages to be produced, and the calculated expected total number of cages according to optimization requirement.

### BACKGROUND

### Technical Field

The present invention relates to an experimental animal managing method, and more particularly, to an experimental animal managing method through an experimental animal management algorithm.

### Background Art

In the field of medical or biological research, animal experiments are conducted on animals to accurately predict the effects of new materials or devices on the human body by confirming the efficacy and safety of new materials or devices.

Various animals such as mice, rabbits, chickens, and dogs are used in animal experiments, among them, mice have the advantage of having a short pregnancy cycle, high fertility, and fast growth rate, so they can secure a large number of individuals, so they are used so much that they account for 80% of domestic experimental animals.

However, when a large amount of animals with high fertility, such as mice, need to be managed in a laboratory, the number of cages accommodating the individuals increases, resulting in a lack of space in experimental animal management facilities.

Also, experimental animals have a delay from mating to use, which causes a mismatch between demand and supply, so it is difficult to systematically manage the breeding space due to difficulty in controlling the appropriate population.

### DETAILED DESCRIPTION OF THE INVENTION

### Technical Problem

In order to solve the above problems, the present invention discloses an experimental animal managing method for managing experimental animals through an experimental animal management algorithm.

### Technical Solution

The present invention was created to achieve the object of the present invention as described above, and discloses an experimental animal managing method comprising:
A receiving step (S10) of receiving user log data and individual data of an experimental animal; and
A processing step (S20) of calculating at least one of an expected production amount of individuals, the expected number of cages to be produced, and the expected total number of cages, on the basis of the user log data and the individual data, and optimizing at least one among the calculated expected production amount of individuals, the calculated expected number of cages to be produced, and the calculated expected total number of cages according to optimization requirement.

The experimental animal managing method according to the present invention may comprise an adjusting step (S30) of adjusting at least one of the number of individuals and the number of cages based on at least one of the expected production amount of individuals, the expected number of cages to be produced, and the expected total number of cages optimized in the processing step (S20).

The receiving step (S10), the processing step (S20), and the adjusting step (S30) may be repeatedly performed at least once.

The optimization requirements are processed in the direction of minimizing at least one of the difference value between the individual demand and the individual supply of the experimental animal, the expected inventory amount of individual and the total number of cages.

The optimization requirements are processed in the direction of maximizing at least one of the production amount of individuals and the variation amount of individuals of the experimental animal.

The processing step (S20) may be performed based on machine learning.

The individual data may include at least one of usage amount of individuals, production amount of individuals, death amount of individuals, inventory amount of individuals, and number of storage spaces of individuals.

At least one of usage amount of individuals, production amount of individuals, death amount of individuals and inventory amount of individuals may be classified according to at least one of the gender of individuals, color of individuals, age of individuals, and management state of individuals.

The expected production amount of individuals is calculated according to
Expected production amount of individuals (R^{weekly})=K·Cₘ·L_{avg},
wherein Cₘ is the number of mating cages (n),
Wherein L_{avg} is the average number of litters (n) born per birth, wherein K is a correction constant, wherein K can be calculated as

$$K = ( DD + 1 ) \times \frac{R}{R + MD} ,$$

Wherein DD (Double Delivery) is the pregnancy success rate (0<DD<1) in the postpartum estrous period, wherein R is the cycle from birth to next birth in the reproduction cycle, wherein assuming that the mean is conception one week after the start of mating, MD (Mating Delay) may be an additionally delayed time (week).

The expected number of cages to be produced is calculated with
Expected number of cages

$$( {R}^{weekly} ) = \frac{K \cdot {C}_{m} \cdot {L}_{avg}}{{M}_{avg}} ,$$

wherein Cₘ is the number of mating cages (n),
wherein L_{avg} is the average number of litters (n) per birth,
wherein K is a correction constant,
wherein M_{avg} is the average number (n) of experimental animals per cage, wherein K can be calculated as

$$K = ( DD + 1 ) \times \frac{R}{R + MD} ,$$

wherein, the DD (Double Delivery) is the pregnancy success rate (0<DD<1) in the postpartum estrous period, wherein R is the cycle from birth to next birth after the reproduction cycle, wherein, assuming that the mean is conception one week after the start of mating, MD (Mating Delay) may be an additionally delayed time (week). The expected total number of cages is calculated according to

$$Expected total number of cages ( {C}_{t , min} ) = \frac{( {T}_{s} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}}$$

when calculating the minimum value, and is calculated according to
Expected total number of cages

$$( {C}_{t , max} ) = \frac{( {T}_{t} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}}$$

when calculating the maximum value,
wherein Tₛ is the minimum age of use of experimental animals,
wherein Tₜ is the maximum age of use of the experimental animal,
wherein T_{w} is the age at which the experimental animal is weaned (weaning: separated from the mother),
wherein K is a correction constant,
wherein Cₘ is the number of mating cages of experimental animals,
wherein L_{avg} is the average number of litters per birth,
wherein M_{avg} is the average number of experimental animals per cage,
wherein R is the cycle from birth to next birth after the reproduction cycle,
wherein Bₛ is the age of the experimental animal at the start of breeding,
wherein Bₜ is the age of the experimental animal at the end of breeding.

### Effects of the Invention

The present invention has the advantage of efficiently managing the inventory of experimental animals and the number of cages required for breeding experimental animals by minimizing the mismatch between demand and supply of experimental animals by providing experimental animal management and an experimental animal managing method using the same.

Also, the present invention derives an optimal value of at least one of an expected production amount of individuals of experimental animal, the expected number of cages to be produced, and the expected total number of cages in the treatment step, on the basis of this, by adjusting the number of individuals and the number of cages, it is possible to increase the experimental animal managing space and production efficiency of experimental animals per unit cage in the laboratory, thereby reducing the cost used for research.

Also, the present invention enables a flexible response to the demand for experimental animals by adjusting the number of individuals and the number of cages by the derived optimal values, and since euthanasia of experimental animals can be minimized by minimizing the inventory of experimental animals, a more ethical laboratory environment can be provided.

Also, the present invention provides the researcher with the optimized number of individuals of experimental animals by adjusting the number of individuals and the number of cages by the derived optimal values, so that the researcher can minimize the effort required to manage the experimental animals.

Moreover, the present invention can be widely applied to optimization of the management of not only experimental animals but also all breeding animals that occupy space, and has an advantage that it can be applied not only to animals but also to inventory managing methods of products in factories.

### BRIEF DESCRIPTION OF THE DRAWINGS

FIG. 1 is a flow chart showing the flow of the experimental animal managing method of the present invention.
S10: receiving step, S20: processing step, S30: adjusting step

FIG. 2 is a graph showing the effect of the experimental animal managing method of the present invention.

FIG. 3 is a table showing the effects of the experimental animal managing method of the present invention.

### DETAILED DESCRIPTION

Hereinafter, an experimental animal managing method according to the present invention will be described with reference to the accompanying drawings.

The present invention discloses an experimental animal managing method for managing experimental animals by an experimental animal management algorithm for systematic and efficient management of experimental animals.

Specifically, the present invention, as shown in FIG. 1, discloses an experimental animal managing method comprising:
A receiving step (S10) of receiving user log data and individual data of an experimental animal; and
A processing step (S20) of calculating at least one of an expected production amount of individuals, the expected number of cages to be produced, and the expected total number of cages, on the basis of the user log data and the individual data, and optimizing at least one among the calculated expected production amount of individuals, the calculated expected number of cages to be produced, and the calculated expected total number of cages according to optimization requirement.

Wherein as the experimental animal, various types of animals such as mice, chickens, dogs, and rabbits may be used, and various genetically modified experimental animals including genetically removed animals and transgenic animals may be used.

Wherein the receiving step (S10) is a step of receiving individual data and user log data to a program installed in a user terminal such as a computer or mobile phone, and various data can be received using a wired/wireless communication network.

Wherein the wired/wireless communication network includes a wired communication network such as Internet or a Public Switched Telephone Network (PSTN), etc., or includes a local area network such as Bluetooth, Zigbee, and RFID communication, etc., and wireless communication networks such as HSPA, 3GPP, 4G and 5G mobile communication networks, etc.

Wherein the individual data is data related to the number of experimental animals and the storage space in which the experimental animals are stored, and may be, for example, data on the usage amount of individual, production amount of individuals, death amount of individuals, inventory amount of individuals, and number of storage space of individuals.

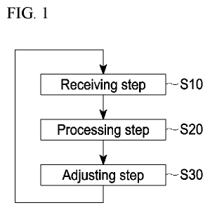

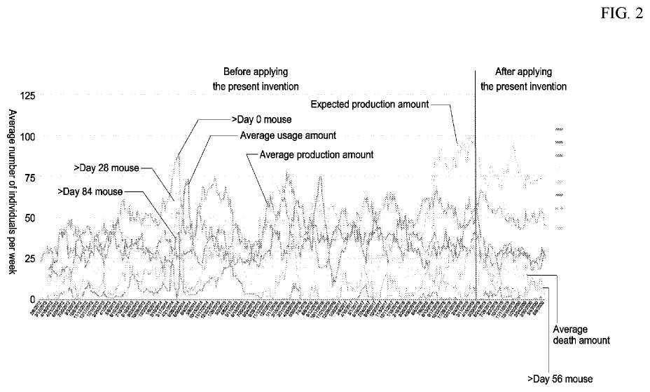

Here, the individual data can be represented as an average data value during a certain period of time so that the trend of change of the individual data can be intuitively confirmed, as shown in FIG. 2, and for example, as an average data value for 8 weeks.

Also, as for the individual data, if the processing step (S20) to be described later is performed based on deep learning, it goes without saying that weekly individual data input values can be averaged in a hidden layer.

Moreover, the usage amount of individual may comprise at least one of the number of experimental animals moving from a specific breeding space to another specific breeding space on average, the number of experimental animals used in the experiment, and the number of experimental animals euthanized after the end of the experiment during a certain period of time.

For example, the usage amount of individual may include at least one of the number of experimental animals moved from the cage where the experimental animals were bred for 8 weeks to the cage where they were moved immediately before the experiment, the number of experimental animals actually used in the experiment, and the number of experimental animals euthanized after the experiment was over.

Wherein, the number of experimental animals moved from a specific breeding space to a specific breeding space during a certain period of time can be definitely used as a leading indicator of the number of experimental animals actually used in the experiment.

The production amount of individuals may be the number of pups born by females on average during a certain period of time, for example, the number of pups born by female experimental animals on average for 8 weeks.

Besides, the death amount of individuals is the number of experimental animals that died on average during a certain period of time, which can be sorted into early death (week 4) and late death (week 12) by time point, and can be sorted into natural death and euthanasia according to the cause of death.

For example, the death amount of individuals may be the number of euthanized individuals, natural death individuals, or the sum of euthanasia and natural deaths in at least one of week 4 (early death) and week 12 (late death).

The inventory amount of individuals is the number of experimental animals of n (n≥0) weeks of age or older that can be used on average per week during a certain period of time, for example, the average of individuals n (n≥0) weeks of age or more that can be used in experiments per week for 8 weeks.

Wherein, the inventory amount of individuals may be increased according to the number of experimental animals obtained by subtracting the death amount of individuals and the usage amount of individuals from the production amount of individuals.

The number of storage space of individuals may be the total number of cages that can be used for at least one of storage, breeding, and mating of experimental animals on average during a certain period of time, for example, it may be the total number of cages in which n (n≥1) experimental animals can be accommodated per cage for a specific time point or 8 weeks.

Here, of course, at least one of the usage amount of individual and the production amount of individuals can be classified according to at least one of the individual gender, individual color, individual age, individual management status, and individual genotype, if necessary.

The user log data is data collected based on researcher's use patterns of experimental animals, and may be configured in various ways.

For example, the user log data may be the data collected the usage amount of individual, usage patterns of individuals of the experimental animal, etc. according to each researcher, time zone, date, experimental animal type, cage used, age of the experimental animal, state of the experimental animal, sex of the experimental animal, etc.

Wherein, the usage amount of individual may be the number of experimental animal investigated by means of permission, order, reservation, etc. before actually using the experimental animal, for example, the data on the usage amount of individual may be obtained through a customer's pre-order, application for use of an experimental animal, a survey on the required amount of an experimental animal, permission to use an experimental animal, etc.

Wherein, the data on the usage amount of individual may be replaced with data obtained from the usage amount of individual described above.

Meanwhile, the individual data and the user log data may be input and stored in a database that systematizes, integrates, and manages the individual data and the user log data for the purpose of sharing and use by multiple people, at this time, a data storage device including the database may be definitely provided.

Here, each data of the individual data and the user log data may be manually or automatically input to a user terminal, that is, a processing device.

For example, the individual data and the user log data can be transmitted to the processing device by each researcher inputting his or her usage record after the user directly checks the individual data such as the state of individuals and number of individuals, etc., as another example, by using an imaging device such as a camera, a sensor device, etc., the imaging device and the sensor device may identify individual data and user log data and transmit them directly to the processing device.

Meanwhile, the individual data and the user log data received in the receiving step (S10) can be used as back data used to calculate the optimized production amount of individuals, euthanasia amount of individuals and the total number of cages required in the processing step (S20).

That is, in the processing step (S20), using the individual data and the user log data, the optimized production amount of individuals, euthanasia amount of individuals, and the total number of cages required are calculated, on the basis of this, by adjusting the total number of cages or the number of cages required for mating and the number of experimental animals, therefore minimizing the mismatch between supply and demand of experimental animals, minimizing inventory of experimental animals, and effectively managing the number of cages required for management of experimental animals.

To this end, in the processing step (S20), based on the individual data and the user log data, at least one of the expected production amount of individuals, the expected number of cages to be produced, and the expected total number of cages is calculated, but at least one of the calculated expected production amount of individuals, the calculated expected number of cages to be produced, and the calculated expected total number of cages may be optimized according to optimization requirements.

Specifically, in the processing step (S20), at least one of the expected production amount of individuals, the expected number of cages to be produced, and the expected total number of cages is calculated using the following formula on the basis of the individual data and the user log data, and an optimal value is learned through simulation so that at least one of the calculated expected production amount of individuals, the calculated expected number of cages to be produced, and the calculated expected total number of cages is optimized according to the optimization requirements, and the above can be performed repeatedly until the optimal value is obtained

First, in the processing step (S20), at least one of the expected production amount of individuals, the expected number of cages to be produced, and the expected total number of cages may be calculated using a formula on the basis of the individual data and the user log data.

At this time, the expected production amount of individuals, the expected number of cages to be produced, and the expected total number of cages are calculated based on a certain period of time, and calculated as the average value of the expected production amount of individuals, the average value of the expected number of cages to be produced, and the average value of the expected total number of cages in a certain period of time.

The certain period (R) may be any period selected according to the needs of the user, but is preferably selected as a cycle from birth to next birth in the reproductive cycle of experimental animals

For example, when a mouse is selected as an experimental animal, the R value may be set to 8 because the reproduction cycle of a female mouse from birth to the next birth is 8 weeks.

The expected production amount of individuals is the number of experimental animals produced on average during a certain period of time, representing an expected increase in supply of experimental animals, and may be calculated as

Expected production amount of individuals (R^{weekly})=K·Cₘ· L_{avg}.

Here, Cₘ is the number (n) of mating cages, L_{avg} is the average number of litters (n) born per birth, and K is a correction constant.

Wherein the correction constant K can be calculated as

$$K = ( DD + 1 ) \times \frac{R}{R + MD} ,$$

wherein, the DD (Double Delivery) is the pregnancy success rate (0<DD<1) in the postpartum estrous period (the period when the number of pups born doubles in the case of successful pregnancy during the fertile period immediately after delivery), wherein R is the cycle from birth to next birth after the reproduction cycle, wherein MD (Mating Delay) is assumed to mean that conception occurs one week after the start of mating, and denotes an additional week of delay (week).

Also, the expected number of cages to be produced required according to the expected production amount of individuals represents the number of cages additionally required according to the expected increase in supply of experimental animals, and can be calculated as Expected number of cages

$$( {R}^{weekly} ) = \frac{K \cdot {C}_{m} \cdot {L}_{avg}}{{M}_{avg}} .$$

Wherein Cₘ is the number (n) of mating cages, wherein L_{avg} is the average number of litters (n) per birth, wherein K is a correction constant as described above, wherein M_{avg} is the average number (n) of experimental animals per cage.
Here, the expected number of cages to be produced may vary depending on the week of use of the experimental animal.

That is, the minimum value of the expected number of cages to be produced is calculated as Expected total number of cages

$$( {C}_{t , min} ) = \frac{( {T}_{s} - {T}_{w} )}{R} \times \frac{K \cdot {C}_{m} \cdot {L}_{avg}}{{M}_{avg}} ,$$

the maximum value of the expected number of cage cages to be produced is calculated as Expected total number of cages

$$( {C}_{t , max} ) = \frac{( {T}_{t} - {T}_{w} )}{R} \times \frac{K \cdot {C}_{m} \cdot {L}_{avg}}{{M}_{avg}} .$$

wherein Tₛ is the minimum age (week) of use of experimental animals,
wherein T_{w} is the age at which the experimental animal is weaned (weaning: separated from the mother), wherein Tₜ is the maximum age (week) of use of the experimental animal, wherein Cₘ is the number (n) of cages used for mating, wherein L_{avg} is the average number of litters (n) per birth, wherein K is a correction constant, wherein M_{avg} is the average number (n) of experimental animals per cage, wherein R is the cycle from birth to next birth after the reproduction cycle.

Therefore, the expected total number of cages required for mating and storing experimental animals on average during a certain period of time is calculated as follows.

That is, the expected total number of cages (C_{t,min}) to have at least is

$${C}_{t , min} = \frac{( {T}_{s} - {T}_{w} )}{R} \times \frac{K \cdot {C}_{m} \cdot {L}_{avg}}{{M}_{avg}} + {C}_{m} ,$$

and the expected total number of cages to have (C_{t,max}) at most is calculated as

$${C}_{t , max} = \frac{( {T}_{t} - {T}_{w} )}{R} \times \frac{K \cdot {C}_{m} \cdot {L}_{avg}}{{M}_{avg}} + {C}_{m} .$$

Here, Cₘ (the number of cages required for mating) can be more specifically calculated by the following formula.

That is, the number of female experimental animals required during a certain period of time (R week) to supplement Cₘ (the number of cages required for mating) is

$${C}_{m} \times \frac{R}{( {B}_{t} - {B}_{s} )} ,$$

and since the number of cages to be produced (C_{mb}) required to maintain this number of female experimental animals is calculated as

$$K \cdot {C}_{mb} \cdot {L}_{avg} = 2 \times {C}_{m} \times \frac{R}{( {B}_{t} - {B}_{s} )} ,$$

it is calculated as

$${C}_{mb} = \frac{2 R}{K \cdot {L}_{avg} \cdot ( {B}_{t} - {B}_{s} )} \times {C}_{m} .$$

Wherein C_{mb} is the number of mating cages for mating,

Wherein Bₛ is the age of the week of the experimental animal to start breeding, and Bₜ is the age of the week of the experimental animal to end the breeding.

That is, if C_{mb} is substituted for Cₘ in the formula of the expected total number of cages, it can be arranged as follows.

The minimum expected total number of cages is

$${C}_{t , min} = \frac{( {T}_{s} - {T}_{w} )}{R} \times \frac{K \cdot {C}_{m} \cdot {L}_{avg}}{{M}_{avg}} + \frac{2 R}{K \cdot {L}_{avg} \cdot ( {B}_{t} - {B}_{s} )} \times {C}_{m} ,$$

again, since Cₘ is needed to maintain Cₘ, the following infinite series formula is obtained.

That is, the minimum value of the expected total number of cages is organized as

$${C}_{t , min} = \frac{( {T}_{s} - {T}_{w} )}{R} \times \frac{K \cdot {C}_{m} \cdot {L}_{avg}}{{M}_{avg}} + {\sum }_{k = 1}^{n} {{C}_{m} ( \frac{2 R}{K \cdot {L}_{avg} \cdot ( {B}_{t} - {B}_{s} )} )}^{k} ,$$

and this can be re-arranged in terms of

$${C}_{t , min} = \frac{( {T}_{s} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}} .$$

Also, the maximum value of the expected total number of cages is

$${C}_{t , max} = \frac{( {T}_{t} - {T}_{w} )}{R} \times \frac{K \cdot {C}_{m} \cdot {L}_{avg}}{{M}_{avg}} + \frac{2 R}{K \cdot {L}_{avg} \cdot ( {B}_{t} - {B}_{s} )} \times {C}_{m} ,$$

which is summarized as and is

$${C}_{t , max} = \frac{( {T}_{t} - {T}_{w} )}{R} \times \frac{K \cdot {C}_{m} \cdot {L}_{avg}}{{M}_{avg}} + {\sum }_{k = 1}^{n} {{C}_{m} ( \frac{2 R}{K \cdot {L}_{avg} \cdot ( {B}_{t} - {B}_{s} )} )}^{k} ,$$

summarized as the formula of

$${C}_{t , max} = \frac{( {T}_{t} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}}$$

Therefore, the expected total number of cages requires a minimum of

$${C}_{t , min} = \frac{( {T}_{s} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}}$$

and a maximum of

$${C}_{t , max} = \frac{( {T}_{t} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}} .$$

In particular, the formulas enable effective management of the production amount of the individual, the number of cages, as an example, the formulas can suggest the ratio of the number of cages required for breeding and the number of cages required for storage in order to maximize the usual amount of the individual in the number of cages secured in the experimental space or to flexibly respond to fluctuations in the usual amount of the individual.

More specifically, the ratio of the number of cages required for breeding and the number of cages required for storage calculated using the formulas can be calculated to have a ratio of 1:1 (in this case, T_{w}=4, Tₜ=8) in the case of maximizing the usual amount of the individual in the number of cages secured in the experimental space, and in the case of flexibly responding to the change in the usual amount of the individual, it can be calculated to have a ratio of 1:2 (in this case, T_{w}=4, Tₜ=12).

Meanwhile, an optimal value is learned through simulation so that at least one of the calculated expected production amount of individuals, the calculated expected number of cages to be produced, and the calculated expected total number of cages by the above formula is optimized according to the optimization requirements.

Here, the optimization requirement is a condition for minimizing a mismatch between supply and demand of experimental animals and increasing the production efficiency of experimental animals per unit cage and management space of experimental animals, and various conditions may be set according to the user's needs.

For example, the optimization requirement may be processed in a direction of minimizing at least one of the difference between the individual demand and the individual supply, the expected inventory amount of individual, the total number of cages, and the researcher's efforts, and may be processed in a direction of maximizing at least one of a production amount of individuals and a fluctuation amount in the number of individuals.

Wherein, the fluctuation amount in the number of individuals is an amount that flexibly changes the number of individuals so that the supply of experimental animals does not run out even when the fluctuation amount in demand of experimental animals is large, for example, even if 10 experimental animals are used in a certain week and 100 experimental animals are used in the following week, it is an amount that fluctuates the number of individuals so that there is no disruption in the supply of experimental animals.

However, in the case where it is not necessary to maximize the amount of change in the number of individuals because the fluctuation range of the individual demand is small, it is definitely possible to optimize in the direction of minimizing the inventory amount of individuals, the total number of cages, and the researcher's efforts.

Also, the optimization requirement may be performed based on machine learning using supervised learning, unsupervised learning, and reinforcement learning, and various programs may be used for this purpose.

Meanwhile, it is possible to effectively manage the experimental individuals by adjusting the number of individuals based on at least one of the optimized expected production amount of individuals, the optimized expected number of cages to be produced, and the optimized expected total number of cages in the processing step (S20).

To this end, the present invention may include an adjusting step (S30) of adjusting the number of experimental individuals and the number of cages according to the optimized expected production amount of individuals, the optimized expected number of cages to be produced, and the optimized expected total number of cages in the processing step (S20).

Wherein the adjusting step (S30) is a step of adjusting the number of individuals based on at least one of the optimized expected production amount of individuals, the optimized expected number of cages to be produced, and the optimized expected total number of cages, and may be performed automatically by having an adjusting device, or may be performed in various ways such as manually performing the adjusting by recognizing an optimum value by the user.

Specifically, the adjusting step (S30), based on at least one of the optimized expected production amount of individuals, the optimized expected number of cages to be produced, and the optimized expected total number of cages, increases or decreases the production amount of individuals by adjusting the time of individual mating, the number of individual mating, etc., or increases or decreases the inventory amount of individuals, or may be adjusted to provide feedback on an optimal value by adjusting the ratio of the number of cages required for mating and the number of cages required for storing experimental animals.

Wherein, the inventory amount of individuals can be adjusted by the production amount of individuals and the user's euthanasia, etc., when the inventory amount of individuals is adjusted by the user's euthanasia, the inventory amount of individuals can be adjusted in response to the demand that immediately changes with the amount of early euthanasia, and may provide an optimized inventory amount of individuals by adjusting the total amount of inventory amount of individuals with the amount of late euthanasia

That is, the adjusting step (S30) adjusts the number of experimental animals and the number of cages according to the optimal value derived from the number of individuals and cages in the experiment space and provides feedback, thereby flexibly responding to the demand of experimental animals with respect to the optimized expected production amount of individuals, the optimized expected number of cages to be produced, and the optimized expected total number of cages, and has the advantage of minimizing the mismatch between demand and supply and of minimizing the inventory of experimental animals.

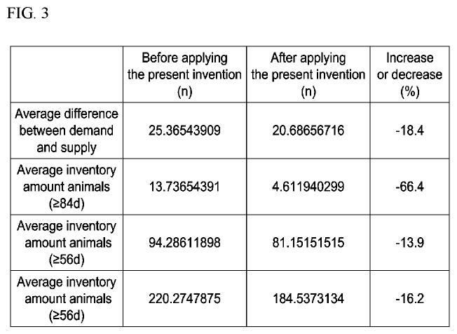

The effect of this invention can be seen by comparing graphs before and after application of the present invention, as shown in FIGS. 2 and 3.

That is, FIGS. 2 and 3 show the difference between demand and supply of an individual and the reducing inventory amount by adjusting the number of individuals according to the optimal value of the present invention when a mouse is selected as the experimental animal.

Specifically, in FIG. 2, the average difference between demand and supply of an individual decreased by about 18.4% from about 25.4 before applying the present invention to about 20.7 after applying the present invention, and the inventory amount of experimental animals also decreased by about 66.4% from about 13.7 to 4.6 animals before and after the application of the present invention in the case of the inventory amount of 84 days or more, and decreased by about 13.9% from about 94.3 to 81.2 before and after application of the present invention in the case of the inventory amount of 56 days or more, and decreased by about 16.2% from about 220.3 to about 184.5 before and after application of the present invention in the case of the inventory amount of 28 days or more, therefore it showed a 32.2% decrease in the average inventory amount of the experimental animals.

That is, the present invention can efficiently manage the inventory of experimental animals and the number of cages necessary for breeding experimental animals by effectively reducing difference between demand and supply of individuals and the inventory amount of experimental animals, therefore the effort and cost for experimental animal management can be minimized.

In the graph of FIG. 2, the number (n) of mice ≥0d and ≥56d was applied as the total number of mice ≥0d and ≥56d for 8 weeks divided by 8 in order to reflect the number of mouse cohorts per week.

This is because, in the case of experimental mice, the minimum week age standard for mice that can be used in experiments is 8 weeks, and since female mice give birth once every 8 weeks, a mouse delivery flow in units of 8 weeks is generated.

Specifically, in the case of experimental mice, since the mouse delivery flow occurs in units of 8 weeks, the number of mice that can be used in equilibrium can be expressed by applying as the number (n) the total number of mice ≥0d, ≥56d for 8 weeks divided by 8, for example, if the production amount of experimental mice is insufficient, immediately the number of mating is increased and after 4 weeks baby mice are produced, as a result, it is possible to recognize the decrease in the inventory amount of experimental mice and respond quickly to maintain equilibrium of the stock.

Also, in order to express the number of experimental animals that can be used in equilibrium as described above, the number (n) dividing the number of experimental animals according to a certain period of time may be differently applied depending on the minimum testable age and the gestation period leading to childbirth, etc. of each experimental animal.

Meanwhile, as shown in FIG. 1, the receiving step (S10), the processing step (S20) and the adjusting step (S30) are processed sequentially the receiving step (S10), the processing step (S20), and the adjusting step (S30), and after the adjusting step (S30), the receiving step (S10) is performed again thereby the receiving step (S10), the processing step (S20), and the adjusting step (S30) may be repeated at least once.

Furthermore, it goes without saying that any one of the receiving step (S10), the processing step (S20) and the adjusting step (S30) may be omitted or performed in a different order.

Moreover, the experimental animal managing method can be widely applied to optimization of management of not only experimental animals but also all breeding animals that occupy space, and may be applicable not only to animals but also to a stock management system for determining stock and factory operation rates in a factory.

Since the above has only been described with respect to some of the preferred embodiments that can be implemented by the present invention, as noted, the scope of the present invention should not be construed as being limited to the above embodiments, and it will be said that the technical idea of the present invention described above and the technical idea together with the root are all included in the scope of the present invention.

What is claimed:

1. An experimental animal managing method comprising:
a receiving step (S10) of receiving user log data and individual data of an experimental animal; and
a processing step (S20) of calculating at least one of an expected production amount of individuals, the expected number of cages to be produced, and the expected total number of cages of the experimental animal, on the basis of the user log data and the individual data, and optimizing at least one among the calculated expected production amount of individuals, the calculated expected number of cages to be produced, and the calculated expected total number of cages according to optimization requirement.

2. The experimental animal managing method according to claim 1, comprising an adjusting step (S30) of adjusting at least one of the number of individuals and the number of cages of the experimental animal based on at least one of the expected production amount of individuals, the expected number of cages to be produced, and the expected total number of cages optimized in the processing step (S20).

3. The experimental animal managing method according to claim 2, wherein the receiving step (S10), the processing step (S20) and the adjusting step (S30) may be repeatedly performed at least once.

4. The experimental animal managing method according to claim 1, wherein the optimization requirement is processed in the direction of minimizing at least one of the difference value between the individual demand and the individual supply of the experimental animal, the expected inventory amount of individual and the total number of cages.

5. The experimental animal managing method according to claim 4, wherein the optimization requirement is processed in the direction of maximizing at least one of the production amount of individuals and the variation amount of individuals of the experimental animal.

6. The experimental animal managing method according to claim 1, wherein the processing step (S20) may be performed based on machine learning.

7. The experimental animal managing method according to claim 1, wherein the individual data comprises at least one of usage amount of individuals, production amount of individuals, death amount of individuals, inventory amount of individuals and number of storage spaces of individuals.

8. The experimental animal managing method according to claim 7, wherein at least one of usage amount of individuals, production amount of individuals, death amount of individuals and inventory amount of individuals may be classified and recognized according to at least one of the gender of individuals, color of individuals, age of individuals, and management state of individuals.

9. The experimental animal managing method according to claim 1, wherein the expected production amount of individuals is calculated according to
Expected production amount of individuals (R^{weekly})=K·Cₘ· L_{avg},
wherein Cₘ is the number of mating cages (n),
wherein L_{avg} is the average number of litters (n) born per birth, wherein K is a correction constant, wherein K can be calculated as
$$K = ( DD + 1 ) \times \frac{R}{R + MD} ,$$
wherein DD (Double Delivery) is the pregnancy success rate (0<DD<1) in the postpartum estrous period, wherein R is the cycle from birth to next birth in the reproduction cycle, wherein MD (Mating Delay) may be an additionally delayed time (week) assuming that the mean is conception one week after the start of mating.

10. The experimental animal managing method according to claim 1, wherein
the expected number of cages to be produced is calculated with
Expected number of cages
$$( {R}^{weekly} ) = \frac{K \cdot {C}_{m} \cdot {L}_{avg}}{{M}_{avg}} ,$$
wherein Cₘ is the number of mating cages (n), wherein L_{avg} is the average number of litters (n) per birth, wherein K is a correction constant, wherein M_{avg} is the average number (n) of experimental animals per cage, wherein K can be calculated as
$$K = ( DD + 1 ) \times \frac{R}{R + MD} ,$$
wherein, the DD (Double Delivery) is the pregnancy success rate (0<DD<1) in the postpartum estrous period, wherein R is the cycle from birth to next birth after the reproduction cycle, wherein, assuming that the mean is conception one week after the start of mating, MD (Mating Delay) may be an additionally delayed time (week).

11. The experimental animal managing method according to claim 1, wherein the expected total number of cages is calculated according to
Expected total number of cages
$$( {C}_{t , min} ) = \frac{( {T}_{s} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}} ,$$
when calculating the minimum value, and is calculated according to when calculating the maximum value,
Expected total number of cages
$$( {C}_{t , max} ) = \frac{( {T}_{t} - {T}_{w} )}{R} \times \frac{K \cdot {C}_{m} \cdot {L}_{avg}}{{M}_{avg}} + \frac{{C}_{m}}{1 - \frac{2 R}{K \cdot {L}_{avg} \cdot ( {B}_{t} - {B}_{s} )}}$$
wherein Tₛ is the minimum age of use of experimental animals, wherein Tₜ is the maximum age of use of the experimental animal, wherein T_{w} is the age at which the experimental animal is weaned (weaning: separated from the mother), wherein K is a correction constant, wherein Cₘ is the number of mating cages of experimental animals, wherein L_{avg} is the average number of litters (n) per birth, wherein M_{avg} is the average number of experimental animals (n) per cage, wherein R is the cycle from birth to next birth after the reproduction cycle, wherein Bₛ is the age of the experimental animal at the start of breeding, wherein Bₜ is the age of the experimental animal at the end of breeding.
